(19) United States
(12) Patent Application Publication
(10) Pub. No.: US 2015/0203432 A1
(43) Pub. Date: Jul. 23, 2015

(54) RECOVERY OF ORGANIC ACIS FROM DILUTE SALT SOLUTIONS

(71) Applicant: ZEACHEM, INC., Lakewood, CO (US)

(72) Inventors: Gal Mariansky, San Francisco, CA (US); Eric Gallegos-West-Ling, Redwood City, CA (US); Dan W. Verser, Menlo Park, CA (US); Timothy J. Eggeman, Lakewood, CO (US); Jesse Coutu, San Francisco, CA (US)

(21) Appl. No.: 14/395,704

(22) PCT Filed: Apr. 22, 2013
(86) PCT No.: PCT/US13/37633

§ 371 Date: Oct. 20, 2014

Related U.S. Application Data
(60) Provisional application No. 61/636,445, filed on Apr. 20, 2012.

(51) Int. Cl. C07C 51/50; C12P 7/06; C07C 51/48; C07C 51/493; C07C 51/44

(52) U.S. Cl. CPC C07C 51/50; C07C 51/493; C07C 51/44; C07C 51/48; C12P 7/06

(57) ABSTRACT

The invention includes a method for recovering an organic acid from a dilute salt solution of the acid, wherein the cation of the salt forms a carbonate salt. The method includes concentrating the solution and combining a tertiary amine, CO2, and a solvent with the solution to form a reaction product medium, an acid/amine complex and a carbonate salt. The acid/amine complex is soluble and the carbonate salt is insoluble in the reaction product medium.

### CROSS-REFERENCE TO RELATED APPLICATION

This PCT application claims priority to U.S. Provisional Patent Application Ser. No. 61/636,445, filed Apr. 20, 2012, entitled “Recovery of Organic Acids from Dilute Salt Solutions.”

### FIELD OF THE INVENTION

The present invention is related to methods for efficiently recovering organic acids from dilute salt solutions, such as fermentation broths.

### BACKGROUND

Organic acids are valuable products as food and feed ingredients, for example, or as intermediates in the production of other chemicals. For example, organic acids can be chemically converted into alcohols, which can subsequently be converted to olefins Such a process could be envisioned as the basis for a biorefinery to convert biomass resources into a range of products for the energy and chemical industries.

Many valuable organic acids, such as acetic, lactic and propionic acids, can be produced by fermentation. Holten, Lactic Acid: Properties and Chemistry of Lactic Acid and Derivatives, Verlag Chemie, 1971; Benninga, (1990), A History of Lactic Acid Making: A Chapter in the History of Biotechnology, Kluwer Academic Publishers, London; Partin, L., Heise, W. (1993), in Acetic Acid and Its Derivatives, Agreda, V., Zoeller, J., ed., Marcel Dekker, New York, pp. 3-13; Playne, 1985 Propionic and butyric acids pp. 731-759, In M. Moo-Young (ed.) Comprehensive Biotechnology, vol. 3, Pergamon, Oxford. Using known fermentation methods, such acids can be produced at very high carbon yield from a wide range of biomass resources. However, today almost all organic acids are produced from petrochemicals.

The production of organic acids by fermentation usually requires neutralization of the broth as fermentation proceeds so that it does not become too acidic. Many fermentation reactions operate optimally near neutral pH and failure to maintain proper control of the pH of the fermentation broth can result in inhibition of the fermentation organism. Thus, maintenance of neutral pH is usually carried out by the addition of a base, such as ammonia, NaOH, Ca(OH)2 or CaCO3, to the fermenter. However, because the cation of the base combines with the organic acid, the result of such treatment is a dilute salt of the organic acid, such as ammonium acetate, sodium acetate or calcium acetate, and not the free acid itself. Also, not all bases are suitable for maintenance of a neutral pH in a fermentation reaction. For example, CaCO₃ is a weak base that only allows pH control in the range of 5-5.5.

Therefore, if it is desired to recover the free acid, it is necessary to convert the organic acid salt back to the free acid, during this conversion the cation of the acid salt, forms a byproduct salt. In order to improve process economics it is desired that the byproduct salt can be recycled back to the fermentation step for pH control. Moreover, these fermentation broths are quite dilute. Thus, an efficient recovery method with respect to both the acidification issue and the dilution issue is desirable.

Many methods have been proposed to address this problem. Among the simplest methods is the addition of a strong mineral acid, such as sulfuric acid, to the broth containing the organic acid salt. Because such acids are much stronger than organic acids, their addition shifts the ionic equilibrium so that essentially all of the organic acid salt is converted to the free acid. However, the strong acid is itself simultaneously converted to a byproduct salt. Because the byproduct salt is formed from the strong acid anion it cannot be recycled to the fermentation step for pH control and if it is not useful it needs to be disposed of. This is an economic and environmental burden since the byproduct salt is produced in an equal molar amount as the organic acid.

Other methods have been proposed to recover the organic acid from the dilute salt solution. One of the more interesting is the use of an amine to convert the alkaline metal salt to an organic salt. For example, Urbas, U.S. Pat. No. 4,405,717, incorporated herein by reference in its entirety, describes the use of tributyl amine (TBA) and CO₂ to convert a dilute calcium salt to an insoluble CaCO₃ and a water-soluble organic complex of TBA and acetic acid at very high yield. Urbas suggests the extraction of the TBA acid complex from the dilute aqueous solution and then the concentration and “cracking” or thermal decomposition of the recovered organic complex to regenerate the TBA and the acetic acid. However this method requires separating the solvent from the amine which is energy intensive. For the extraction step Urbas teaches away from the use of solvents like alcohols that react with the acid and recommends the use of chloroform which is problematic because of its toxicity to the environment.

Verser et al. (U.S. Patent Publication No. 2005/0256337), incorporated herein by reference in its entirety, describe the recovery of the acid from the extracted TBA acid complex by forming its ester directly from the extract. However, the esterification reaction is conducted in the presence of the amine and is fairly slow.

Similarly, Verser et al. (U.S. Pat. No. 6,509,180), incorporated herein by reference in its entirety, describe the production of ethanol from acetic acid produced by fermentation. The acetic acid is reacted with an amine to form an acid/amine complex, which is then thermally cracked to release the acid. The released acid is then esterified to form alcohol. Similarly, Verser et al. (U.S. Patent Publication No. 20080193989), incorporated herein by reference in its entirety, also teaches forming a complex between an organic acid and an amine.

Mariansky et al. (U.S. Patent Publication No. 2009/0281354 A1), incorporated herein by reference in its entirety, describe the recovery of the acid from a TBA acid complex by thermally cracking the complex at high temperature while extracting the TBA into a solvent phase. The acid, now in the protonated form, can be recovered from the dilute aqueous solution by a second extraction. This recovery process requires two extraction trains, one of them operated at high temperature and high pressure, which make this process too costly for a commercial process.

A number of other processes have been proposed for recovery of organic acids from dilute acid salt solutions. Thomas et al. published U.S. Patent Application 2006/0024801 A1 where they reacted the salt with a low molecular amine, concentrate by evaporation, replace the low molecular amine with a high molecular amine in an extractive distillation column and distill the acid from the high molecular amine. Similar to Mariansky et al., this process requires two extraction trains and is too costly for a commercial process.

Verser et al. (PCT Publication No. WO 2012/054400 A1), incorporated herein by reference in its entirety, describe a process where a calcium salt of acetic acid is reacted with carbon dioxide and an amine to form a soluble acid/amine complex and insoluble CaCO₃ salt in the present of a water immiscible solvent. The acid/amine complex is extracted into the solvent phase which is then distilled to recover the acid. Similar to the patents above by Verser, Mariansky and Urbas the reaction of the acetic acid with the amine is pushed to high yields by the formation of an insoluble CaCO₃ salt. However, CaCO₃ is a weak base that cannot be used for control pH in most organic acid fermentation processes and therefore cannot be recycled directly back to the fermenter.

King, et al. U.S. Pat. No. 5,068,180, describe a method to recover the acid by adsorbing it on a strongly basic ion exchanger and desorbing using a light amine or ammonia solution. The resulting salt is then thermally cracked by evaporating the amine and water away from this acid. This method offers only limited recovery from salt solutions and only works for non-volatile acids (e.g. lactic acid)

Baniel et al. U.S. Pat. No. 6,087,532, describe an extraction method to recover the acid from a salt solution by combining it with high molecular weight amine and CO₂. The carbonic acid resulting from the CO₂ dissolving in the water acidifies a portion of the acid salt which is then extracted into the amine phase. Because most organic acids are stronger acids than carbonic acid, only a small portion of the acid salt is acidified and therefore the extraction coefficient is very low which necessitates a high solvent to feed ratio for high recovery rates of the acid.

Datta et al. U.S. Pat. No. 5,723,639 teaches a method where a light amine or ammonia and a light alcohol are combined with the salt solution; the mixture is heated in the presence of a catalyst and subjected to pervaporation with hydrophilic membrane. However the reaction rates and conversion are too low to be practical in a commercial process.

Thus, while the prior art discloses methods for recovering organic acids from fermentation broths, such methods require two separate extraction loops or high solvent to feed ratios or high energy use, or all of these combined. Another issue with most prior art is that the reaction of the acid salt with the amine and CO₂ produces CaCO₃ as a by-product. CaCO₃ is a weak base that cannot be used to control pH in most organic acid fermentation processes and therefore cannot be recycled directly back to the fermenter. This necessitates disposing of the CaCO₃ and purchasing Ca(OH)₂ for fermentation pH control or using a lime kiln to convert the CaCO₃ to Ca(OH)₂, both are expensive options.

Thus, a need exists for a simple method that provides a process with low capital cost, low energy use, allows recovery of the acid in a concentrated form from the dilute acid salt and produces by-products that can be directly recycled to the fermenter. The present invention satisfies this need and provides other advantages as well.

### BRIEF DESCRIPTION OF THE FIGURES

FIG. 1 is a process flow diagram illustrating a process for converting sodium acetate into ethanol using methods of the present invention.

FIG. 2 is a process flow diagram representing the second part of the process illustrated in FIG. 1.

FIG. 3 is a process flow diagram illustrating a process for converting potassium acetate into acetic acid using methods of the present invention.

FIG. 4 illustrates the reactor temperature during carbonation of potassium acetate with acetone and tributylamine.

FIG. 5 illustrates the amount of potassium acetate in the initial fermentation converted into potassium carbonate by carbonation of potassium acetate with acetone and tributylamine.

FIG. 6 illustrates the distribution coefficient for acetic acid obtained at various concentrations of acetic acid using two different solvent extraction systems. Diamonds represent the distribution coefficient obtained using a solvent system containing 40% hexanol/60% hexyl acetate. Squares represent the distribution coefficient obtained using a solvent system containing 60% hexanol/40% hexyl acetate.

### SUMMARY OF THE INVENTION

The present innovation provides an efficient method for the recovery and separation of organic acids (e.g., carboxylic acids) from a dilute solution such as a fermentation broth, where the organic acids are in the form of salts formed from reaction of the acid and a base used to neutralize the acid during fermentation for pH control.

One embodiment of the present invention is a method for recovering an organic acid from a dilute salt solution of the acid, wherein the cation of the salt forms a carbonate salt, comprising: concentrating the dilute salt solution, and combining a tertiary amine, CO₂, and a solvent with the concentrated solution to form a reaction product medium, an acid/amine complex and a carbonate salt, wherein the acid/amine complex is soluble and the carbonate salt is insoluble in the reaction product medium.

In one embodiment, the dilute salt solution is obtained by fermentation. In one embodiment, the concentration of the organic acid salt in the solution is lower than the saturation concentration of the organic acid salt in water at 60° C., and not more than 20% lower than the saturation concentration in water at 20° C. Concentration of the dilute salt solution may be achieved using any suitable method. In one embodiment, the step of concentrating is selected from the group consisting of evaporation, nanofiltration, reverse osmosis, dialysis, adsorption of water on a water-selective adsorbent, extraction of water into a water-selective extractant and combinations thereof.

The acid being recovered can be any acid. In one embodiment, the acid has a boiling point greater than the boiling point of the solvent. In one embodiment, the acid is a carboxylic acid. In one embodiment, the acid is acetic acid, lactic acid, propionic acid, butyric acid, succinic acid, citric acid, 3-hydroxypropionic acid, glycolic acid or formic acid.

The salt of such acids may comprise any cation that is capable of forming a carbonate-containing salt that is insoluble in the reaction product. In one embodiment, the solubility of the carbonate salt in the solvent is less than 2000 ppm. In one embodiment, the cation is Ca, Na, K, NH₄, Zn, Ba or Mg. In one embodiment, the cation is not calcium.

The present method uses amines to form an acid/amine complex. In one embodiment, the amine is a tertiary amine. In one embodiment, the tertiary amine is soluble in the solvent. In one embodiment, the tertiary amine is selected from the group consisting of tributylamine, dicyclohexyl methyl amine, di-isopropyl ethyl amine, tripropylamine and mixtures thereof. In one embodiment, the solubility of the acid/amine complex is at least 5 wt % pm the acid basis.

In one embodiment, the step of forming the acid/amine complex is conducted at a temperature in the range of about 10° C. to about 65° C. In one embodiment, the step of forming the acid/amine complex is conducted at a pressure in the range of about 25 psig to about 350 psig. In one embodiment, the ratio of solvent to the carboxylic acid is in the range of about 0.3 to about 1.5 by mass.

The present method uses solvents to recover the acid. Any solvent may be used so long as it works with the chosen amine to effect recovery of the acid from the solvent phase. In one embodiment, the solvent is miscible in water at room temperature. In one embodiment, the boiling point of the solvent is lower than the boiling point of the organic acid and the amine. In one embodiment, the solvent is selected from the group consisting of low molecular weight alcohols, low molecular weight ketones, low molecular weight ethers and mixtures thereof. In one embodiment, the solvent is selected from the group consisting of acetone, tetrahydrofuran, methanol, ethanol, isopropanol, and mixtures thereof.

In some embodiments, the insoluble carbonate salt is removed from the reaction product medium. In one embodiment, the insoluble carbonate salt is removed from the solution by a solid/liquid separation. In one embodiment, the solid/liquid separation is selected from the group consisting of gravity separation, filtration, centrifugation and combinations thereof.

In some embodiments, the solvent is removed from the reaction product medium In one embodiment, removal of the solvent comprises distilling the solvent from the solution in an overhead product and forming a bottoms product comprising the tertiary amine, the acid/amine complex and water. In a further embodiment, the bottoms product forms a top and a bottom phase, wherein the bottom phase comprises the acid/amine complex. In a further embodiment, the bottom phase is subjected to liquid-liquid extraction to produce an extract comprising the acid/amine complex. In one embodiment, the extract is dehydrated to produce a dry extract. In one embodiment, the dry extract is distilled to produce a vapor phase comprising the liquid-liquid extraction solvent and the organic acid. In one embodiment, the vapor phase is subjected to reactive distillation to form a product between the liquid-liquid extraction solvent and the organic acid. In one embodiment, the product of the claimed method is an ester. In a further embodiment, the ester is subjected to hydrogenolysis to form an alcohol of the organic acid and the liquid-liquid extraction solvent.

In one embodiment, solvent is removed from the reaction product medium by flash distilling the solution to produce a vapor stream and a liquid stream, wherein the vapor stream contains solvent and the liquid stream comprises water, solvent residue and the acid/amine complex. In a further embodiment, the liquid stream is dried to produce a distillate comprising solvent and water and a bottom stream comprising the dry acid/amine complex. In a further embodiment, the acid/amine complex is thermally treated to produce a vapor phase comprising the acid and a bottom stream comprising the amine. In a further embodiment, a product is produced from the acid/amine complex. In one embodiment, the product is selected from the group consisting of an organic acid, an ester and an alcohol.

One embodiment of the present invention is a method to produce an organic acid, comprising: a) culturing a microorganism in a medium to produce a solution comprising the organic acid; b) adding a base to the medium, wherein the cation of the base forms an insoluble carbonate salt, and is not calcium; c) concentrating the medium; and, combining a tertiary amine, CO₂, and a solvent with the medium to form an acid/amine complex and the insoluble carbonate salt. In one embodiment, the step of adding a base raises the pH of the medium to a pH that is better tolerated by the microorganism.

One embodiment of the present invention is a method for recovering an organic acid from a dilute salt solution of the acid, wherein the cation of the salt forms a carbonate salt, comprising: a) concentrating the dilute salt solution; b) combining a tertiary amine, CO₂, and a solvent with the concentrated solution to form a reaction product medium, an acid/amine complex and a carbonate salt, wherein the acid/amine complex is soluble and the carbonate salt is insoluble in the reaction product medium; and, c) recovering the organic acid from the reaction product medium. In one embodiment, the step of recovering the organic acid comprises thermally dissociating the acid/amine complex to produce free acid and amine In one embodiment, prior to dissociation of the acid/amine complex, the reaction product medium is distilled to produce a vapor stream and a liquid stream, wherein the vapor stream contains solvent and the liquid stream comprises water, solvent residue and the acid/amine complex. In a further embodiment, prior to dissociation of the acid/amine complex, the method further comprises drying the liquid stream. In one embodiment, prior to dissociation of the acid/amine complex, the method further comprises removing the insoluble carbonate salt from the reaction product medium.

### DETAILED DESCRIPTION OF THE INVENTION

The present innovation provides an efficient method for the recovery and separation of organic acids (e.g., carboxylic acids) from a dilute solution such as a fermentation broth, where the organic acids are in the form of salts formed from reaction of the acid and a base used to neutralize the acid during fermentation for pH control.

The production of organic acids by fermentation usually results in acidification of the fermentation medium. However, many fermentations operate optimally near a neutral pH and failure to maintain proper pH control of the fermentation broth results in inhibition of the fermentation organism. Consequently, neutralization of the broth during fermentation is necessary so that the broth does not become too acidic. Thus, maintenance of a neutral pH can be carried out by addition of a base such as ammonia, NaOH, Ca(OH)₂ or CaCO₃. However, not all bases are suitable for maintenance of a neutral pH in a fermentation reaction. For example, CaCO₃ is a weak base that only allows pH control in the range of 5-5.5.

The present innovation provides an efficient method for the recovery and separation of organic acids (e.g., carboxylic acids) from a dilute solution such as a fermentation broth, where the organic acids are in the form of salts formed from reaction of the acid and a base used to neutralize the acid during fermentation for pH control. For example, if a fermentation producing acetic acid (HAc) is neutralized with calcium hydroxide, the resulting organic acid salt produced in fermentation will be calcium acetate (Ca(Ac)₂):

2HAc+Ca(OH)₂→Ca(Ac)₂+2H₂O

The organic acid salt can then be reacted with an amine, such as a tertiary amine such as tributylamine (TBA), and carbon dioxide to form an acid/amine complex and an insoluble carbonate salt. For example:

Ca(Ac)₂+H₂O+CO₂+2TBA=>2TBA:HAc+CaCO₃

While not being bound by theory, it is believed that the carbonation reaction is pulled to the right by precipitation of the CaCO₃ salt, which results in very high conversions of the acid salt to an acid/amine complex. However, since CaCO₃ is a very weak base, when recycled back to the fermentation, it only allows limited pH control in the range of 5-5.5. The disclosed method allows for improved pH control in fermentation reactions used to produce dilute solutions of organic acids. As such, the present innovation provides a process that allows, but does not require, replacing calcium with other cations such as sodium, potassium or ammonium in the carbonation reaction. For example:

NaAc_{(aq)}+H₂O₍ₗ₎+CO_{2(g)}+TBA₍ₗ₎=>TBA:HAc_{(aq)}+NaHCO₃₍ₛ₎

or

KAc_{(aq)}+H₂O₍ₗ₎+CO_{2(g)}+TBA₍ₗ₎=>TBA:HAc_{(aq)}+KHCO₃₍ₛ₎

or

NH₄Ac_{(aq)}+H₂O₍ₗ₎+CO_{2(g)}+TBA₍ₗ₎=>TBA:HAc_{(aq)}+NH₄HCO₃₍ₛ₎

The bicarbonate salts produced in the carbonation reactions above possess sufficient base strength to allow fermentation pH control in the neutral pH range. While not being bound by theory, it is believed that the carbonation reaction is pulled to the right by precipitation of the bicarbonate salt. However, since the bicarbonate salts of sodium, potassium and ammonium are much more soluble than calcium carbonate in aqueous solutions, such reactions give very low product yields. In accordance with the present innovation it has been surprisingly found that the reactions above can be pushed to very high yields by concentrating the dilute organic acid salt and conducting the reaction in the presence of a solvent. For example:

KAc_{(aq)}+H₂O₍ₗ₎+CO_{2(g)}+TBA₍ₗ₎+C₃H₆O₍ₗ₎)=>TBA:HAc_{(aq)}+KHCO₃₍ₛ₎+C₃H₆O₍ₗ₎

In the reaction above, potassium acetate is converted to an acetic acid/tributylamine complex and potassium bicarbonate. Potassium bicarbonate is soluble in aqueous solutions; however it is insoluble in the solvent-water solution of this reaction, so the product of this reaction is an acid/amine complex dissolved in the acetone-water solution and insoluble potassium bicarbonate salt. Without being bound by theory it is believed that the solvent, acetone in this example, acts as a solvent for the acid/amine complex but as an anti-solvent for potassium bicarbonate and therefore causes the potassium bicarbonate to precipitate. Because of the precipitation of the potassium bicarbonate salt, the reaction can be pulled the right and give very high yields that were previously only attainable with organic acid salt of calcium.

The present invention provides a method for recovering an organic acid from a dilute salt solution of the acid. The method includes concentrating the dilute salt solution and combining a tertiary amine, carbon dioxide and a solvent with the dilute salt solution to form an insoluble carbonate-containing salt and an acid/amine complex. In further embodiments of the innovation, the acid is recovered from the mixture.

The acid being recovered can be any organic acid. In a preferred embodiment, the acid has a boiling point greater than the boiling point of the solvent. In one embodiment, the acid is a carboxylic acid. In one embodiment, the acid is acetic acid, lactic acid, propionic acid, butyric acid, succinic acid, citric acid, 3-hydroxypropionic acid, glycolic acid, formic acid or mixtures thereof.

Methods of the present invention are particularly suited to the recovery of products produced by fermentation. In various embodiments, the fermentation medium includes carbohydrate substances, non-carbohydrate substances, and mixtures thereof. Carbohydrate in the fermentation medium can be obtained from biomass, which can include, but is not limited to herbaceous matter, agricultural residue, forestry residue, municipal solid waste, waste paper, pulp and paper mill residue. Biomass can also be selected from the group consisting of trees, shrubs, grasses, wheat, wheat straw, wheat midlings, sugar cane bagasse, corn, corn husks, corn kernel, corn fiber, municipal solid waste, waste paper, yard waste, branches, bushes, energy crops, fruits, fruit peels, flowers, grains, herbaceous crops, leaves, bark, needles, logs, roots, saplings, short rotation woody crops, switch grasses, vegetables, vines, sugar beet pulp, oat hulls, hard woods, wood chips, intermediate streams from pulping operations and soft woods, and in a preferred embodiment, is selected from the group consisting of trees, grasses, whole plants, and structural components of plants.

The biomass can be pre-treated prior to fermentation. For example, if an agricultural product such as corn is used as a carbohydrate source, the corn can be ground to produce corn meal and/or oil for recovery. In one embodiment, the biomass is hydrolyzed to produce carbohydrate prior to fermentation. In one embodiment, the hydrolysis is enzymatic hydrolysis.

In one embodiment, the fermentation is conducted using a microorganism that is a homofermentative microorganism, and can be selected from homoacetogenic microorganisms, homolactic microorganisms, propionic acid bacteria, butyric acid bacteria, succinic acid bacteria and 3-hydroxypropionic acid bacteria. In one embodiment, the fermentation is conducted using a microorganism that produces acetate as the primary end product of metabolism. In other embodiments, the microorganism is of a genus selected from Clostridium, Lactobacillus, Moorella, Thermoanaerobacter, Propionibacterium, Propionispera, Anaerobiospirillum, and Bacteroides. In other embodiments, the microorganism is of a species selected from Clostridium formicoaceticum, Clostridium thermoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteroides amylophilus and Bacteroides ruminicola.

Reaction of the cation of the organic acid salt with carbonic acid from the CO₂ results in formation of carbonate-containing salt that is insoluble in the reaction product medium (i.e., the concentrated salt solution and solvent mixture). As has been discussed, it is believed that precipitation of the carbonate-containing salt drives formation of the acid/amine complex, thereby increasing the efficiency of recovery of the acid. Thus, any cation that is capable of forming a carbonate-containing salt that is insoluble in the reaction product solution can be used in the present method. Suitable cations include, for example, Ca, Na, K, NH₄, Zn, Ba and Mg. In a preferred embodiment, the cation is selected from the group consisting of Ca, Na, K and NH₄, and in another preferred embodiment, the cation is selected from the group consisting of Na, K and NH₄. In one embodiment, the cation is a cation other than calcium.

The preferred salt concentration of the concentrated organic acid salt varies according to the salt used. In one embodiment, the concentration of the organic acid salt is lower than the acid salt saturation concentration in water at 60° C. In another embodiment, the concentration of the organic acid salt is not more than 20% lower than the saturation limit at 20° C. In a preferred embodiment, the concentration of the organic acid salt is lower than the acid salt saturation concentration in water at 60° C. and is not more than 20% lower than the saturation limit at 20° C. For example, for KAc dissolved in water, the concentrated salt solution entering the reaction vessel will preferably contain less than 77 wt % KAc and more than 58 wt % KAc. For KAc dissolved in fermentation broth that contains other ions, the saturation concentration will be lower and therefore the concentrated salt solution entering the reactor will have a lower concentration. Without being bound by theory, it is generally preferred to have the least amount of water in the reaction solution in order to reduce the solubility of the carbonate-containing salt while avoiding taking out too much water and precipitating the organic acid salt.

According to the present invention, any suitable method can be used to concentrate the dilute salt solution. Suitable concentration methods include, but are not limited to, evaporation, nanofiltration, reverse osmosis, dialysis, adsorption of water on a water selective adsorbent, extraction of water into a water-selective extractant, and combinations thereof.

The present method utilizes an amine in order to form an acid/amine complex. In one embodiment, the amine is a tertiary amine. In one embodiment, the amine is selected from the group consisting of tributylamine, dicyclohexyl methyl amine, di-isopropyl ethyl amine, tripropylamine, and mixtures thereof.

Depending on the acid being recovered and the amine chosen for the process, the acid/amine complex will have varying coefficients of solubility and extractability. Thus, the choice of solvent will be affected by the amine used in, and the acid recovered by, the present method. Any solvent can be used so long as it works with the chosen amine to effect recovery of the acid from the solvent phase. The main function of the solvent is to act as an anti-solvent to the base (e.g., NaHCO₃/KHCO₃/NH₄HCO₃). Another important function of the solvent is to dissolve the produced acid/amine complex. Preferred solvents are those having properties that improve the yield and efficiencies of the disclosed method. For example, in one embodiment, the solvent is miscible with water at room temperature. In one embodiment, the tertiary amine and the CO₂ are soluble in the solvent. In one embodiment the acid/amine complex has solubility in the solvent that is at least 5 wt % on the acid basis. In one embodiment, the carbonate-containing salt has a solubility in the solvent of less than 2000 ppm. In one embodiment, the solvent has a boiling point that is lower than the boiling point of the acid and the boiling point of the amine. Preferred ratios of solvent to concentrated organic salt solutions are in the range of about 0.3 to about 1.5 by mass. In a further embodiment, the solvent is polar.

In one embodiment, the solvent is selected from the group consisting of low molecular weight alcohols, low molecular weight ketones, low molecular weight ethers and mixtures thereof. In one embodiment, the solvent is selected from the group consisting of acetone, tetrahydrofuran, methanol, ethanol, isopropanol and mixtures thereof.

As noted above, the cation reacts with carbonic acid formed by the CO₂, resulting in the formation of a carbonate-containing salt that is insoluble the reaction product medium. Precipitation of this salt is believed to improve the efficiency of the process since removal of the cation from the reaction mixture drives formation of the acid/amine complex. As used herein, the term carbonate-containing salt includes any salt of carbonic acid, bicarbonate, carbonate or mixtures thereof. A carbonate-containing salt is also referred to as a carbonate salt.

The step of combining a tertiary amine, CO₂, and solvent with the dilute salt solution of the acid can be conducted at any suitable temperature and pressure. The reaction is preferably conducted at temperatures in the range of about 10° C. to about 65° C. and pressures in the range of about 25 psig to about 350 psig. With the solvent present in the concentrated system, the conversion to the carbonate-containing salts can be rapid and can proceed to very high conversion as the carbonate-containing salt is precipitated from the mixture by the solvent.

In a further embodiment, the insoluble carbonate-containing salt is separated from the reaction mixture by a solid/liquid separation. In a particular embodiment, the insoluble carbonate-containing salt is separated from the solvent. Any method that separates the insoluble carbonate-containing salt from the reaction mixture can be utilized. Methods of separation are known to those skilled in the art and include, but are not limited to, for example, gravity separation, filtration, centrifugation, and combinations thereof.

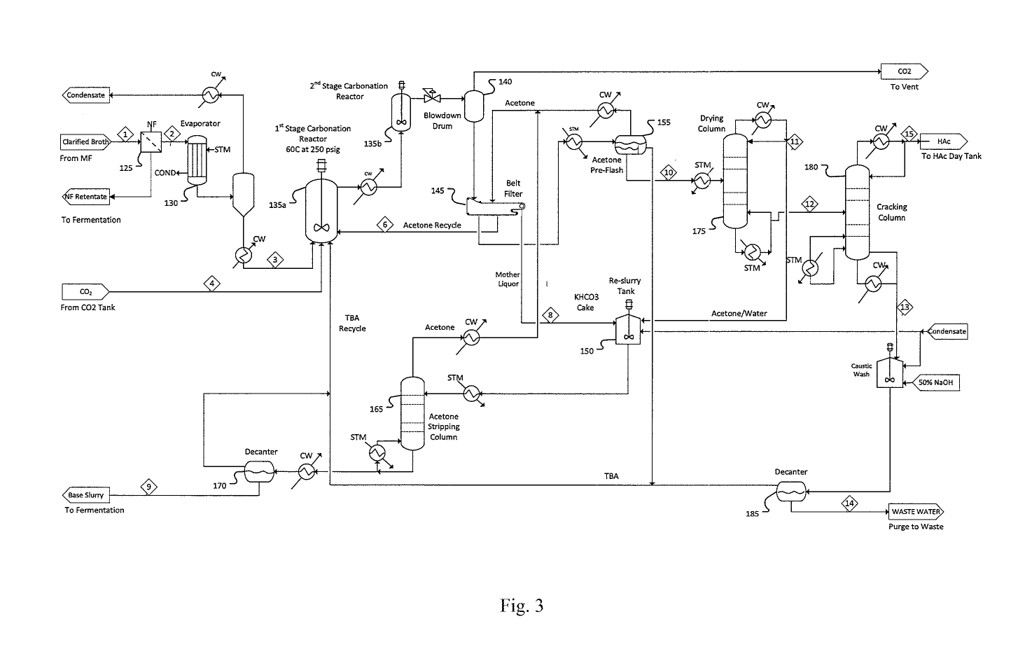

In one preferred embodiment, after the solids are removed, the solvent is removed from the solution and can be recycled to the carbonation step. Such removal can be by volatilization of the solvent, such as by distillation (e.g., flashing), to produce a vapor stream containing the solvent and a liquid stream comprising water, solvent residue and the acid/amine complex. The liquid stream may be dried (e.g., by distillation) and the acid/amine complex thermally dissociated (e.g., cracked) in a second distillation column to produce a concentrated organic acid overhead product stream and concentrated amine bottom stream that is recycled to the carbonation reactor. A preferred embodiment of this process is described below with reference to FIG. 3.

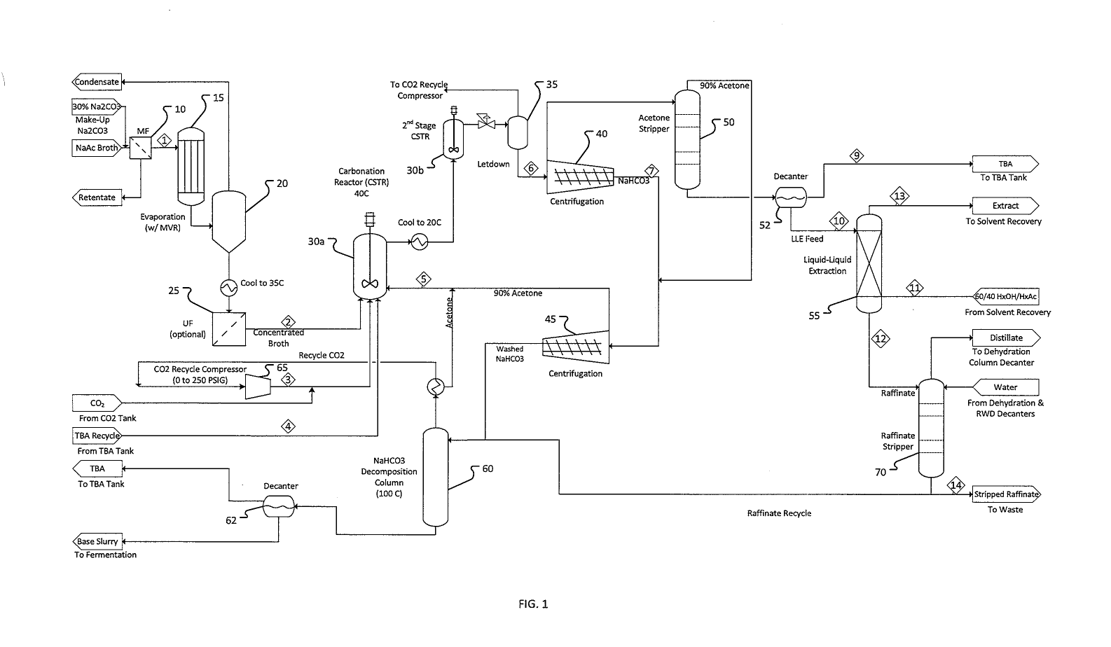

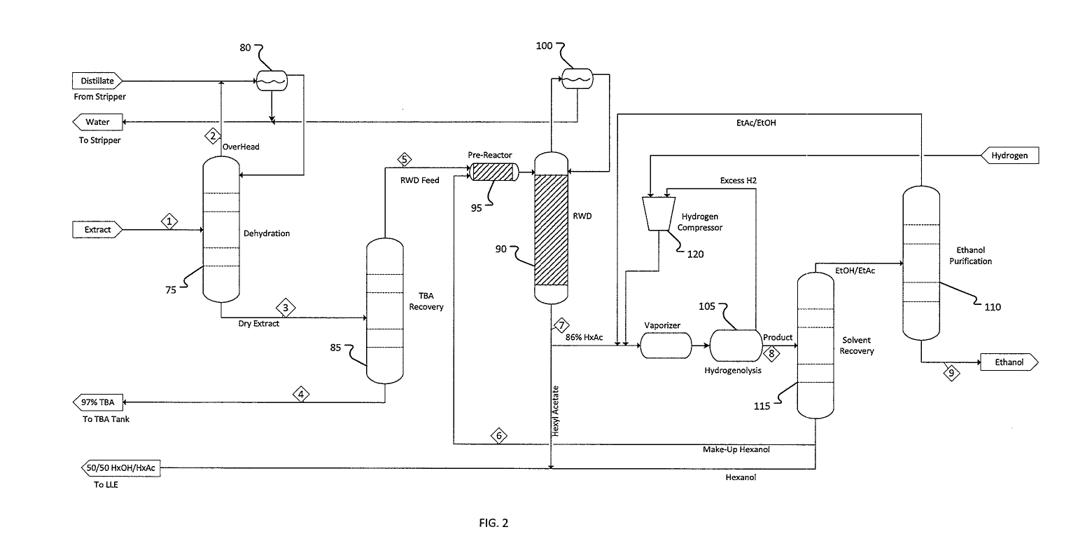

An alternative embodiment following removal of the solids is described below with reference to FIGS. 1 and 2. The separated carbonate-containing solid stream can be diluted with water and sent to a stripping column to remove residual solvent. The dilution of the solids with water causes any residual amine to salt out, and it can be recovered by decantation after the solvent removal step. The carbonate-containing salt solution is recycled to fermentation for pH control. Alternatively the carbonate-containing salt solution can be heated to temperatures above 100° C. which will cause the carbonate-containing salt to decompose to the carbonate species, which increases the base strength and base solubility of the solution.

The current innovation offers several advantages over prior art. One such advantage is the ability to use K, Na and NH₄ based systems. The result of being able to use such systems is that the bicarbonate produced in the carbonation reaction can be directly recycled to the fermenter and can be used to control pH in the 6-7 range which is suitable for most organic acid fermentation. In a system using calcium as the cation, as disclosed in prior art, the resulting carbonate salt needs to be converted to calcium hydroxide in a lime kiln before it can be recycled to the fermenter for pH control in the 6-7 range.

Another advantage is that potassium, sodium and ammonium systems offer much higher solubility than a calcium system; this reduces fouling in heat exchangers and allows concentrating the dilute fermentation broth to high concentrations without solids precipitation which reduces fouling in heat exchangers. Higher concentration also reduces downstream equipment size.

Another advantage is that the process described in this innovation is much simpler in terms of equipment count and operability than prior art processes for recovery of organic acid from dilute salt solutions.

In the following section two specific applications of the current innovation are given which show how the innovation can be used in practice.

The following examples are provided for the purpose of illustration and are not intended to limit the scope of the present invention.

### Examples

### Example 1

### Process Description of Carbonation, Extraction and Complex Cracking from Dilute Sodium Acetate with Acetone and Tributylamine

This example is a process to produce 25 MM gallons of ethanol per year from a dilute sodium acetate (NaAc) solution that was produced in fermentation. The process description is given with reference to FIGS. 1 and 2. Sodium acetate fermentation broth is treated in a microfiltration unit 10 to remove the biomass and then fed to a mechanical vapor recompression evaporator 15 and 20 (stream 1, FIG. 1). The evaporation stage removes about 80% of the water in the broth which is concentrated to 31 wt % NaAc. The concentrated broth can optionally be treated by ultrafiltration 25.

The concentrated broth (stream 2, FIG. 1) is fed to the carbonation reactor 30a, 30b along with acetone (stream 5, FIG. 1), CO₂ (stream 3, FIG. 1) and tributylamine (TBA) (stream 4, FIG. 1). The products of the reaction are a soluble tributyl amine:acetic acid (TBA:HAc) complex and sodium bicarbonate (NaHCO₃) solids suspended in the acetone-water solution. The carbonation reaction train includes two CSTR reactors 30a, 30b running at 250 psig with cooling between the two reactors. The reaction product proceeds to a blowdown tank 35 where the pressure is reduced to atmospheric. CO₂ from the blowdown tank is recycled back to a CO₂ compressor and the liquid product is fed (stream 6, FIG. 1) to a centrifuge 40 to separate the NaHCO₃ solids (stream 7, FIG. 1) from the TBA:HAc liquid stream.

The NaHCO₃ solids (stream 7, FIG. 1) proceed to the washing centrifuge 45 and the TBA:HAc liquid stream is fed to an acetone stripping column 50 which operates at atmospheric pressure. In the acetone stripper 50, the acetone is taken as 90 wt % acetone in water overhead product which is sent to the washing centrifuge 45 to be used for NaHCO₃ washing. The bottoms product of the acetone stripper 50 is split to two phases in a liquid/liquid separator 52, the top phase contains about 20% of the TBA fed (stream 9, FIG. 1) to the carbonation reactor 30a, 30b, and is recycled back to the TBA tank. The bottom phase containing the rest of the TBA, HAc and water goes forward (stream 10, FIG. 1) to the liquid-liquid extraction (LLE) column 55.

The NaHCO₃ solids stream (stream 7, FIG. 1) is mixed with the acetone overhead stream from the acetone stripping column 50. The resulting slurry is centrifuged and the liquid phase is recycled to the carbonation reactor 30a, 30b. The solids stream from the washing centrifuge 45 is diluted with water and fed to the top of a decomposition column 60 operating at slightly elevated pressure. Live steam is fed at the bottom of the column 60. In the column 60, most of the NaHCO3 is decomposed to make Na2CO₃ and CO₂. This reaction is pushed forward to about 85% conversion by stripping the CO₂ with steam. The overhead product containing steam, acetone and CO₂ is condensed. The CO₂ is recycled to a CO₂ compressor 65 and the acetone-water mixture is recycled to the carbonation reactor 30a. The bottom product from the column comes out as two liquid phases which are separated in a liquid/liquid separator 62. The top phase containing mainly TBA is recycled to the TBA tank. The bottom phase, which is practically free of TBA, contains dissolved Na₂CO₃ and a small amount of NaCO₃:NaHCO₃:2H₂O (Trona) solids is recycled to the fermentation area for pH control.

The TBA:HAc aqueous stream from the acetone stripper 50 (stream 10, FIG. 1) is fed to the top of the LLE column 55 and a solvent composed of a mixture of 60/40 wt % hexanol (HxOH) and hexyl acetate (HxAc) is fed to the bottom of the column (stream 11, FIG. 1). The column 55 is mechanically mixed (e.g. Karr or pulsed column) and operates at atmospheric pressure and 70° C. The solvent flows counter-currently to the aqueous feed and extracts TBA and HAc from it. The rich solvent phase (the extract) (stream 13, FIG. 1) is sent to the solvent recovery section and the depleted aqueous phase (the raffinate) (stream 12, FIG. 1) is sent to the raffinate stripper 70.

In the raffinate stripper 70, residual TBA and hexyl acetate are steam stripped as low boiling heterogeneous azeotropes. The overhead stream is condensed and cooled and fed to a decanter where two liquid phases form. The top phase is used as reflux in the dehydration column and the bottom phase is recycled to the stripper. The bottoms stream from the stripper 70 is split, ˜40% is sent to waste and ˜60% is recycled and used to dilute the NaHCO₃ solids stream going into the NaHCO₃ decomposition column 60.

The extract from the LLE column 55 (stream 1, FIG. 2) is fed to a dehydration column 75. In the dehydration column 75, water is taken overhead as low boiling heterogeneous azeotropes of water/hexanol and water/hexyl acetate. The overhead stream (stream 2, FIG. 2) is sent to the raffinate stripper decanter 80, and the organic phase from that decanter is returned as reflux. The bottom stream (stream 3, FIG. 2) from the dehydration column is fed to the TBA recovery column 85. In the TBA recovery column 85, the solvent plus acetic acid are distilled from TBA and are fed to the Reaction with Distillation (RWD) column 90. The TBA bottoms stream is recycled to the TBA tank. Both the dehydration 75 and TBA recovery 85 columns operate at atmospheric pressure.

In the RWD section, HAc is reacted to completion with excess HxOH to make HxAc according to the following esterification reaction:

HxOH+HAc←→HxAc+H₂O

The HxOH/HxAc/HAc feed mixture (stream 5, FIG. 2) from the TBA recovery section is combined with additional HxOH (stream 6, FIG. 2) recycled from the hydrogenation section to increase the ratio of HxOH to HAc to 1.3 by mole. The mixture is fed to a catalytic packed bed reactor 95 containing strong acid cation exchange resin as the catalyst. In the reactor, the reaction is taken to ˜60% completion. The reactor product is fed to the top of the RWD column 90. The RWD column 90 operates at atmospheric pressure or light vacuum and utilizes the same catalyst as the reactor in the reaction zone. As HxOH, HxAc and HAc go down the column, water produced by the esterification reaction is removed as HxOH/H₂O and HxAc/H₂O low boiling azeotropes. This allows the reaction to go to completion at the bottom of the column. The bottoms product containing 88/12 wt % HxAc/HxOH mixture goes forward to the hydrogenation section. The overhead stream is condensed, cooled and fed to a decanter 100. The top phase from the decanter is used as reflux and the bottom phase is sent to the raffinate stripper 70.

Upstream of the hydrogenation reactor 105, the feed from the RWD section (stream 7, FIG. 2) is mixed with an ethyl acetate/ethanol (EtAc/EtOH) recycle stream from the ethanol purification column 110, pumped to reactor pressure (˜200 psig), mixed with the hydrogen feed stream from a hydrogen compressor 120, heated to about 215° C. and vaporized. The catalytic isothermal hydrogenation reactor 105 converts the HxAc to ethanol and hexanol. Small amounts of EtAc are produced as a byproduct. In the HxOH recovery column 115 the hexanol from the hydrogenation product stream (stream 8, FIG. 2) is recovered as the bottom stream and EtOH and EtAc are taken as overhead. This column operates at atmospheric pressure. In the EtOH purification column 110 EtAc/EtOH is taken overhead and concentrated ethanol is taken as the bottom product stream (stream 9, FIG. 2). This column operates at 30 psig.

### Example 2

### Process Description of Carbonation and Complex Cracking from Dilute Potassium Acetate with Acetone and Tributylamine

This example is for a process to produce 100 Kilotons per annum (KTA) glacial acetic acid from a dilute potassium acetate (KAc) solution that was produced in fermentation. The process description is given with reference to FIG. 3. Potassium acetate fermentation broth is microfiltered to remove the bio mass and then fed to a nano filtration unit 125. In the nano filtration unit 125, sugars and fermentation media components are retained and recycled back to fermentation. The dilute KAc (stream 2, FIG. 3) continues forward to a mechanical vapor recompression evaporator 130. The evaporation stage removes about 96% of the water in the broth which is concentrated to 73 wt % KAc.

The concentrated broth (stream 3, FIG. 3) is fed to the carbonation reactor 135a, 135b along with acetone (stream 6, FIG. 3), CO₂ (stream 4, FIG. 3) and tributylamine (TBA) (stream 5, FIG. 3). The products of the reaction are a soluble tributyl amine:acetic acid (TBA:HAc) complex and potassium bicarbonate (KHCO₃) solids suspended in the acetone-water solution. The carbonation reaction train includes two CSTR reactors 135a, 135b running at 250 psig with cooling between the two reactors. The reaction product proceeds to a blowdown tank 140 where the pressure is reduced to atmospheric. CO₂ from the blowdown tank 140 is vented or recycled back to a CO₂ compressor and the liquid product is fed to a belt filter 145 to separate and wash the KHCO₃ solids (stream 7, FIG. 3) from the TBA:HAc liquid stream.

The KHCO₃ solids (stream 8, FIG. 3) proceed to the re-slurry tank 150 and the TBA:HAc liquid stream is fed to an acetone flash unit 155 which operates at atmospheric pressure. In the acetone flash, most of the acetone is taken as 93 wt % acetone in water distillate product which is sent to the belt filter to be used for KHCO₃ cake washing. The liquid product of the acetone flash splits to two phases, the top phase contains ˜15% of the TBA fed to the carbonation reactor 135a, 135b, and is recycled back to the carbonation reactor 135a, 135b. The bottom phase containing the rest of the TBA, HAc and water goes forward to the drying column 160.

The KHCO₃ solids stream (stream 8, FIG. 3) is re-slurried with condensate water from the evaporator 175. The resulting slurry is heated and fed to the top of the acetone stripping column 165 which operates at a slightly elevated pressure. In the column, acetone is stripped from the solids mixture. The bottom product from the column comes out as two liquid phases in a decanter 170. The top phase containing mainly TBA is recycled to the TBA tank. The bottom phase, which is practically free of TBA, contains dissolved KHCO₃ and a small amount KHCO₃ solids is recycled to the fermentation area for pH control.

The wet TBA:HAc stream (stream 10, FIG. 3) is dried in an atmospheric drying column 175. The distillate stream contains acetone and water and is recycled to the re-slurry tank 150. The bottom stream containing dry TBA:HAc (stream 12, FIG. 3) is fed to the cracking column 180. The cracking distillation column 180 operates at atmospheric pressure. The top stream contains concentrated acetic acid that can be sold as a product or further processed into other product, e.g. ethanol (see first example). The bottom concentrated TBA stream (stream 13, FIG. 3) is cooled and washed with caustic to remove any impurities from the TBA. After the caustic washing the stream splits to two phases in a decanter 185. The top TBA phase is recycled to carbonation and the bottom TBA-free aqueous stream is sent to waste.

### Example 3

### Carbonation of Potassium Acetate with Acetone and Tributylamine

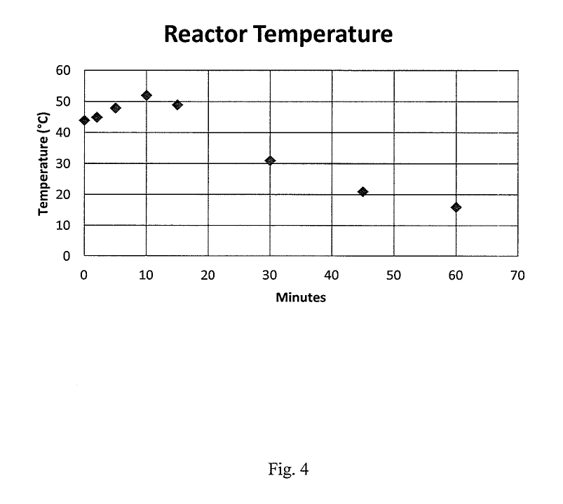

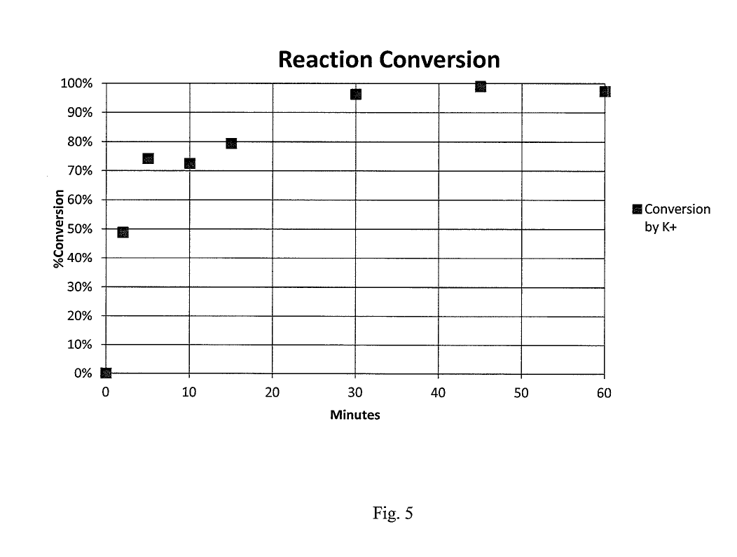

The kinetics of the carbonation reaction using potassium acetate (KAc) containing fermentation broth was measured in a batch reactor using acetone as the anti-solvent and TBA as the amine. Micro filtered fermentation broth containing KAc was concentrated to 66 wt % KAc by batch evaporation. 801 grams of 66 wt % KAc broth, 747 grams of TBA, 63 grams of water and 723 grams of acetone were charged into a 2 gallon high pressure stirred PARR reactor. After the reactor headspace was pressurized to 250 psig, the mixture agitated at 525 RPM, and CO2 sparged through the bottom of the reactor at 6.2 lb/hr. The agitation and CO2 sparging was started simultaneously and commenced timing of the reaction. A back pressure valve was utilized to keep the reactor pressure at 250 psig. Samples were taken after 0, 2, 5, 10, 15, 30, 45 and 60 minutes. After 10 minutes the reactor was cooled with chilled water. FIG. 4 shows the reactor temperature over time. The samples were centrifuged to separate the solid and the liquid phases and the amount of K+ in the liquid phase determined by ion chromatography. After 60 minutes, the reactor was depressurized and the solids were separated by filtration and rinsed with a mixture of 93% acetone and 7% water. The amount of K+ present in the rinse was determined by ion chromatography. The percent conversion of K+ was calculated using Equation 1 (below) and verified by acetic acid analysis of the liquid phase and the rinse fraction described above. FIG. 5 shows the percent conversion as a function of time. After 30 minutes 98.5% conversion was achieved; the temperature of the slurry at this time was 31° C.

$$\% Conversion = 1 - \frac{Mass KAc in Slurry}{Mass KAc in Initial Broth} \qquad Equation 1$$

### Example 4

### Carbonation of Different Organic Acids Salts

This example illustrates the effects of different cations, anti-solvents, acids and anti-solvent to aqueous feed ratios on the carbonation reaction conversion. Each experiment was performed with similar procedures and operating conditions as described in Example 3. Results are shown in Table 1.

TABLE 1
SUMMARY OF REACTION CONVERSION

| | | Anti- | Aqueous: | | |
|---|---|---|---|---|---|
| | Aqueous | Solvent | Anti- | % Conversion | % Conversion |
| wt % Organic Acid Salt | Feed | Feed | Solvent | at 20° C. | at 30 minutes |
| 27.5% Sodium Acetate | Evaporated | Acetone | 1.00 | 97.1% | 96.4% |
| | Broth | | | | |
| 30.8% Sodium Acetate | Evaporated | Acetone | 0.65 | 91.3% | 93.2% |
| | Broth | | | | |
| 38.5% Ammonium Acetate | Synthetic | Acetone | 1.00 | 92.2% | 87.4% |
| 21.2% Calcium Acetate | Evaporated | Methanol | 0.30 | n/a | 99.6% |
| | Broth | | | | |
| 21.2% Calcium Acetate | Evaporated | Ethanol | 0.30 | 98.8% | 98.8% |
| | Broth | | | | |
| 13.8% Potassium Acetate, | Evaporated | Acetone | 0.90 | 99.1% | 98.1% |
| 38.7% Potassium Propionate, | Broth | | | | |
| 3.96% Potassium Succinate | | | | | |
| 15.3% Potassium Acetate, | Synthetic | Acetone | 0.90 | 99.5% | 99.3% |
| 53.7% Potassium Propionate | | | | | |
| 72.2% Potassium Acetate | Evaporated | Acetone | 0.50 | 91.0% | 88.4% |
| | Broth | | | | |
| 66% Potassium Acetate | Evaporated | Acetone | 0.90 | 99.0% | 98.5% |
| | Broth | | | | |
| 75% Potassium Acetate | Synthetic | Acetone | 0.50 | 99.8% | 99.7% |
| 75% Potassium Acetate | Synthetic | Acetone | 0.90 | 99.7% | 99.7% |
| 75% Potassium Acetate | Synthetic | Acetone | 1.10 | 99.5% | 99.6% |

### Example 5

### Solubility of Bicarbonates in Anti-Solvents

To select the best anti-solvent for carbonation, the solubility of potassium bicarbonate and sodium bicarbonate was determined in several solvent-water systems. Ellingboe and Runnels (“Solubilities of Sodium Carbonate and Sodium Bicarbonate in Acetone-Water and Methanol-Water Mixtures”, Journal of Chemical and Engineering Data 1966, 11, 323-324) studied the solubility of sodium bicarbonate in methanol-water and acetone-water mixtures. Platonov (Platonov, A. Y., et al., “Solubility of Potassium Carbonate and Potassium Hydrocarbonate in Methanol”, Journal of Chemical and Engineering Data 2002, 47, 1175-1176) reported the solubility of potassium bicarbonate in pure methanol. The solubility of potassium bicarbonate in acetone-water mixtures was measured by saturating the mixtures and determining the amount of K+ in the resulting liquid phase by ion chromatography. The results for these studies are summarized in Table 2. The results show that solubility of both bicarbonate salts are drastically reduced as the wt % of the solvent in the mixture is increased. The results also show that the solubility of both bicarbonate salts is less in acetone than in methanol.

TABLE 2
BICARBONATE SOLUBILITY IN DIFFERENT SOLVENT SYSTEMS

| | | Solubility | | | |
|---|---|---|---|---|---|
| | | (wt % | wt % Anti- | wt % | Temp |
| Salt | Solvent System | Salt) | Solvent | water | (° C.) |
| NaHCO3 | Water | 10.02% | 0% | 89.98% | 22 |
| NaHCO3 | Methanol/Water | 4.13% | 42.37% | 53.50% | 22 |
| NaHCO3 | Methanol/Water | 3.07% | 82.12% | 14.81% | 22 |
| NaHCO3 | Methanol | 2.13% | 97.87% | 0.00% | 22 |
| NaHCO3 | Acetone/Water | 1.57% | 43.50% | 54.93% | 22 |
| NaHCO3 | Acetone/Water | 0.02% | 89.98% | 10.00% | 22 |
| NaHCO3 | Acetone | 0.02% | 99.98% | 0.00% | 22 |
| KHCO3 | Water | 25.21% | 0% | 74.79% | 20 |
| KHCO3 | Methanol | 0.02% | 99.98% | 0.00% | 25 |
| KHCO3 | Acetone/Water | 0.03% | 50% | 49.97% | 25 |
| KHCO3 | Acetone/Water | 0.003% | 80% | 20.00% | 25 |
| KHCO3 | Acetone/Water | 0.001% | 99.999% | 0.00% | 25 |

### Example 6

### Purification of Acetic Acid without Extraction

This example demonstrates the following steps of the process described in Example 2: acetone flashing by batch evaporation, dehydration by batch distillation and complex cracking by batch distillation. These steps are used to recover the concentrated acid from the acid/amine complex produced in the carbonation step.

Acetone flashing: 711 grams of a liquid mixture containing by weight 8% water, 19.2% HAc, 42% TBA and 30.8% acetone was prepared. The mixture was charged into a 5 L round bottom flask and heated at atmospheric pressure with an electric mantle. The vapors were condensed, collected in an overhead receiver and the condensate analyzed to determine the percentage of each component. The mixture was evaporated until the bottoms temperature reached 97.4° C. and the vapor temperature reached 87.9° C. The resulting distillate weighed 212.6 grams and had the following composition by weight: 14.3% water, 0.1% HAc, 1.4% TBA and 83.9% acetone. The bottom separated into a heavy phase and a light phase. The light phase weighed 86.56 grams and had the following composition by weight: 1.6% water, 2.8% HAc, 88% TBA and 7.6% acetone; the heavy phase weighed 399.4 grams and had the following composition by weight: 6.2% water, 33.4% HAc, 52% TBA and 8.5% acetone.

Dehydration: 398.4 grams of the bottoms heavy phase from the previous acetone flash test was charged into a 3 L round bottom flask fit with a ten inch long distillation column packed with ProPak® high-efficiency packing and an overhead condenser. The flask was heated using an electric mantle and the distillation run with total reflux until the vapor temperature stabilized. Distillate was drawn from the condenser receiver with partial reflux until the overhead temperature reached 108° C. The resulting distillate weighed 64.8 grams and had the following composition by weight: 39.7% water, 6.6% HAc, 1.6% TBA and 52.2% acetone. The bottoms weighed 333.6 grams and had the following composition by weight: 38.6% HAc and 61.7% TBA.

Complex cracking: 333.6 grams of the bottoms from the previous dehydration test was charged into a 1 L round bottom flask fit with a ten inch long distillation column packed with Pro-Pak® high-efficiency packing and an overhead condenser. The flask was heated using an electric mantle and the distillation run with total reflux until the vapor temperature stabilized. Distillate was drawn from the condenser receiver with partial reflux until the bottoms temperature reached 213.6° C. The resulting distillate weighed 109.6 grams and had the following composition by weight: 99.99% HAc and 0.004% TBA. The bottoms weighed 221.5 grams and had the following composition by weight: 0.1% HAc and 99.9% TBA.

### Example 7

### Extraction of Acid-Amine Complex

This example demonstrates the liquid-liquid extraction step described in Example 1. An aqueous mixture of 28 wt % sodium acetate was carbonated with acetone and TBA in a batch reactor using a procedure similar to that described in Example 3, resulting in the production of slurry containing sodium bicarbonate and the TBA/acetic acid complex. The solids were removed from the slurry and the acetone removed from the liquid portion using the procedure described in Example 4, yielding a mixture having the following composition by weight: 48.6% water, 16.9% HAc, 33.4% TBA and 1.0% acetone. This mixture was used as the aqueous feed in the following extractions.

The aqueous feed was placed in a 50 ml conical tube with a solvent containing 40% hexanol and 60% hexyl acetate at a solvent to feed ratio (S/F) of 0.6. The tube was incubated in a 60° C. water bath for one hour, after which the temperature of the two phases was measured to ensure they were at 60° C., and the tube shaken vigorously and placed back in the hot water bath to allow separation of the liquid phases. Both liquid phases were transferred to individual containers, weighed and analyzed for HAc and TBA. Fresh solvent was added to the raffinate at the same S/F and the process was repeated. In total, five cross-current extractions were performed. The distribution coefficient (K_{d}) of HAc was calculated for each extraction using Equation 2:

$${K}_{d} = \frac{wt \% HAc in Extract}{wt \% HAc in Raffinate} \qquad Equation 2$$

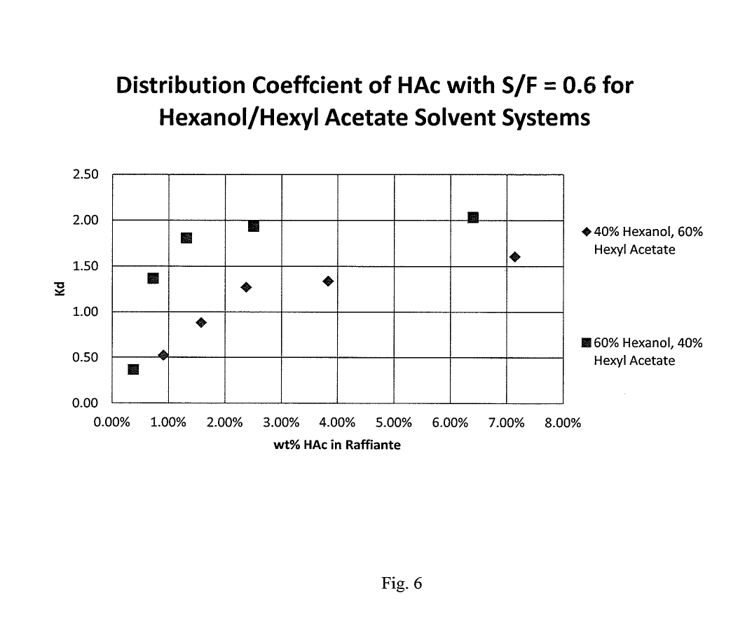

The procedure was repeated using a solvent system containing 60% hexanol and 40% hexyl acetate with the same S/F of 0.6. FIG. 6 shows the K_{d} as a function of wt % HAc in the raffinate for each extraction.

### Example 8

### Recovery of Bicarbonate Salts from Carbonation Slurry

A carbonation slurry obtained from an experiment similar to that described in Example 3 was analyzed to determine the percent of each component. The solid portion contained 21.5% KHCO3 by weight. The liquid portion of the slurry had the following composition by weight: 14.6% water, 16.8% HAc, 35.5% TBA, 0.23% KAc and 33% acetone. A 716 gram sample of the well-mixed slurry was filtered using a 20 micron filter cloth, resulting in a cake weighing 256 grams, having a moisture content of 29.4% and containing 6.4 wt % HAc. The cake was then washed with 181.2 grams of a 92% acetone/8% water mixture leaving a cake that weighed 217.9 grams, having a moisture content of 17.8% and containing 3.2 wt % HAc. Each part of the filtration was timed and the total filtration capacity was 1040 kg dry solids per square meter per hour. No solids breakthrough was observed.

In a separate experiment, 368.5 grams of washed potassium bicarbonate was re-slurried with 331.6 grams of water in a 1 L round bottom flask fitted with a condenser and electric heating mantle. The flask was heated and the condensed overhead collected until the temperature of the bottoms fraction reached 90° C. The solids completely dissolved once the mixture reached 70.8° C. and the final bottoms separated into a heavy phase and a light phase. The bottoms heavy phase weighed 565.3 grams and had the following composition by weight: 52.7% water, 0.36% acetone and 46.9% KHCO3. No TBA was detected in the bottoms heavy phase. The bottoms light phase weighed 10.22 grams and had the following composition by weight: 61.24% TBA and 38.0% HAc.

### Example 9

### Cracking of TBA:HAc Complex in Extract with Hexanol as the Solvent

In this example a mixture representing a dehydrated extract containing the TBA:HAc complex in hexanol is distilled in a batch column to test if the complex can be cracked in the presence of hexanol. A mixture weighing 2009 grams and having a composition by weight of 10% HAc, 30.7% TBA and 59.3% hexanol, was charged into a 3 L round bottom flask comprising a heating mantle, a packed column and a condenser. The mixture was heated and run with total reflux until the overhead temperature stabilized at about 113° C. Distillate was then collected while maintaining some partial reflux until the vapor temperature reached 156.6° C. The distillate weighed 567.7 grams and had the following composition by weight 5.94% water, 12.7% HAc, 0.04% TBA, 81.4% hexanol and 0.3% hexyl acetate. The bottoms weighed 1440 grams and had the following composition by weight 0.18% water, 0.02% HAc, 44.2% TBA, 44.3% hexanol and 17.6% hexyl acetate.

1. A method for recovering an organic acid from a dilute salt solution of the acid, wherein the cation of the salt forms a carbonate salt, comprising:
a. concentrating the solution; and
b. combining a tertiary amine, CO₂, and a solvent with the solution to form a reaction product medium, an acid/amine complex and a carbonate salt, wherein the acid/amine complex is soluble and the carbonate salt is insoluble in the reaction product medium.

2. The method of claim 1, wherein the concentration of the organic acid salt in the solution is lower than the saturation concentration of the organic acid salt in water at 60° C., and not more than 20% lower than the saturation concentration in water at 20° C.

3. (canceled)

4. (canceled)

5. (canceled)

6. (canceled)

7. The method of claim 1, wherein the tertiary amine and CO₂ are soluble in the solvent.

8. The method of claim 1, wherein the boiling point of the solvent is lower than the boiling point of the organic acid and the amine.

9. (canceled)

10. (canceled)

11. The method of claim 1, further comprising separating the insoluble carbonate salt from the solution by a solid/liquid separation.

12. (canceled)

13. The method of claim 11, further comprising removing the solvent from the solution.

14. The method of claim 13, wherein the step of removing comprises distilling the solvent from the solution in an overhead product and forming a bottoms product comprising the tertiary amine, the acid/amine complex and water.

15. The method of claim 14, wherein the bottoms product forms a top and a bottom phase, wherein the bottom phase comprises the acid/amine complex.

16. The method of claim 15, further comprising subjecting the bottom phase to liquid-liquid extraction to produce an extract comprising the acid/amine complex.

17. (canceled)

18. (canceled)

19. The method of claim 16, further comprising forming a product between the liquid-liquid extraction solvent and the organic acid.

20. The method of claim 19, wherein the product is an ester.

21. The method of claim 20, further comprising subjecting the ester to hydrogenolysis to form an alcohol of the organic acid and the liquid-liquid extraction solvent.

22. The method of claim 13, wherein the step of removing comprises flash distilling the solution to produce a vapor stream and a liquid stream, wherein the vapor stream contains solvent and the liquid stream comprises water, solvent residue and the acid/amine complex.

23. The method of claim 21, further comprising drying the liquid stream to produce a distillate comprising solvent and water and a bottom stream comprising the dry acid/amine complex.

24. The method of claim 22, further comprising thermally treating the acid/amine complex to produce a vapor phase comprising the acid and a bottom stream comprising the amine.

25. The method of claim 1, further comprising producing a product from the acid/amine complex.

26. (canceled)

27. (canceled)

28. (canceled)

29. (canceled)

30. (canceled)

31. (canceled)

32. (canceled)

33. (canceled)

34. A method to produce an organic acid, comprising:
a. culturing a microorganism in a medium to produce a solution comprising the organic acid;
b. adding a base to the medium in order to raise the pH of the medium to a pH that is better tolerated by the microorganism, wherein the cation of the base forms an insoluble carbonate salt, and is not calcium;
c. concentrating the medium;
d. combining a tertiary amine, CO₂, and a solvent with the medium to form an acid/amine complex and the insoluble carbonate salt.

35. (canceled)

36. A method for recovering an organic acid from a dilute salt solution of the acid, wherein the cation of the salt forms a carbonate salt, comprising:
a. concentrating the dilute salt solution;
b. combining a tertiary amine, CO₂, and a solvent with the concentrated solution to form a reaction product medium, an acid/amine complex and a carbonate salt, wherein the acid/amine complex is soluble and the carbonate salt is insoluble in the reaction product medium; and,
c. recovering the organic acid from the reaction product medium.

37. The method of claim 36, wherein the step of recovering the organic acid comprises thermally dissociating the acid/amine complex to produce free acid and amine.

38. The method of claim 37, wherein prior to dissociation of the acid/amine complex, the method further comprises distilling the reaction product medium to produce a vapor stream and a liquid stream, wherein the vapor stream contains solvent and the liquid stream comprises water, solvent residue and the acid/amine complex.

39. (canceled)

40. (canceled)
